(19) United States
(12) Patent Application Publication
(10) Pub. No.: US 2024/0188874 A1
(43) Pub. Date: Jun. 13, 2024

(54) BIOLOGICAL STATE ESTIMATION DEVICE

(71) Applicant: OMRON HEALTHCARE CO., LTD., Muko-shi (JP)

(72) Inventors: Akito ITO, Kyoto (JP); Kenji FUJII, Kyoto (JP); Yasuhiro KAWABATA, Kyoto (JP); Naomi MATSUMURA, Kyoto (JP); Yuki SAKAGUCHI, Kyoto (JP)

(73) Assignee: OMRON HEALTHCARE CO., LTD., Muko-shi (JP)

(21) Appl. No.: 18/582,237

(22) Filed: Feb. 20, 2024

Related U.S. Application Data
(63) Continuation of application No. PCT/JP2022/031743, filed on Aug. 23, 2022.

(30) Foreign Application Priority Data
Sep. 1, 2021 (JP) 2021-142288

(51) Int. Cl. A61B 5/349; A61B 5/00; A61B 5/0245

(52) U.S. Cl. CPC A61B 5/349; A61B 5/0245; A61B 5/7221

(57) ABSTRACT

A biological state estimation device that estimates a biological state based on at least an electrocardiographic feature amount and comprises an electrocardiographic waveform acquisition unit; an electrocardiographic feature amount extraction unit, a pulse waveform acquisition unit; a pulse wave feature amount extraction unit an electrocardiographic waveform quality determination unit; a first search reference point setting unit for setting a first reference point for searching the electrocardiographic waveform when the electrocardiographic waveform is of quality to enable extraction of the electrocardiographic feature amount; and a second search reference point setting unit that sets a second reference point for searching the electrocardiographic waveform on the basis of the pulse wave feature amount when the electrocardiographic waveform is of quality to enable extraction of the electrocardiographic feature amount. The electrocardiographic feature amount extraction unit searches the electrocardiographic waveform based on the first reference point or the second reference point an.

### CROSS-REFERENCE TO RELATED APPLICATIONS

This application is the U.S. national stage application filed pursuant to 35 U.S.C. 365(c) and 120 as a continuation of International Patent Application No. PCT/JP2022/031743, filed on Aug. 23, 2022, which application claims priority to Japanese Patent Application No. 2021-142288, filed on Sep. 1, 2021, which applications are incorporated herein by reference in their entireties.

### TECHNICAL FIELD

The present invention relates to a biological state estimation device.

### BACKGROUND ART

Conventionally, there has been known a device that simultaneously measures an electrocardiographic waveform and a pulse waveform and estimates a biological state from a feature amount, such as a heartbeat interval, related to a circulatory organ.

In such a device that simultaneously measures an electrocardiographic waveform and a pulse waveform, a feature amount is basically extracted from the electrocardiographic waveform, but when a large amount of noise is superimposed on the electrocardiographic waveform, supplementation is performed with a feature amount obtained from the pulse waveform (see Patent Literature 1).

### CITATION LIST

### Patent Literature

Patent Literature 1: JP 2009-261419 A

### SUMMARY OF INVENTION

### Technical Problem

However, in such a conventional technique, since the electrocardiographic waveform is not searched in a case where a large amount of noise is superimposed on the electrocardiographic waveform, it is difficult to extract the feature amount existing only in the electrocardiographic waveform by using the pulse waveform.

In view of the conventional technique as described above, an object of the present invention is to provide a technique capable of searching an electrocardiographic waveform on the basis of a feature amount of a pulse waveform to accurately extract a feature amount of the electrocardiographic waveform even in a case where there is a noise that is likely to affect the electrocardiographic waveform.

### Solution to Problem

In order to solve the above problems, the present invention is
a biological state estimation device which includes:
   an electrocardiographic waveform acquisition unit which acquires an electrocardiographic waveform;
   an electrocardiographic feature amount extraction unit which searches the electrocardiographic waveform and extracts a feature amount of the electrocardiographic waveform;
   a pulse waveform acquisition unit which acquires a pulse waveform; and
   a pulse wave feature amount extraction unit which searches the pulse waveform obtained by the pulse waveform acquisition unit and extracts a feature amount of the pulse waveform, and
which estimates a biological state on the basis of at least the feature amount of the electrocardiographic waveform among the feature amount of the electrocardiographic waveform and the feature amount of the pulse waveform, the biological state estimation device further including:
   an electrocardiographic waveform quality determination unit which determines whether or not the electrocardiographic waveform has a quality of being able to extract the feature amount of the electrocardiographic waveform from only the electrocardiographic waveform;
   a first search reference point setting unit which sets a first reference point for searching the electrocardiographic waveform in a case where it is determined that the electrocardiographic waveform has the quality of being able to extract the feature amount of the electrocardiographic waveform; and
   a second search reference point setting unit which sets a second reference point for searching the electrocardiographic waveform on the basis of the feature amount of the pulse waveform in a case where it is determined that the electrocardiographic waveform does not have the quality of being able to extract the feature amount of the electrocardiographic waveform,
in which the electrocardiographic feature amount extraction unit searches the electrocardiographic waveform on the basis of the first reference point or the second reference point and extracts the feature amount of the electrocardiographic waveform.

According to this, even when it is determined that the electrocardiographic waveform has a quality of being unable to extract the feature amount of the electrocardiographic waveform, the electrocardiographic waveform is searched for with reference to the second reference point set on the basis of the feature amount of the pulse waveform. Thus, even in a case where there is noise that is likely to affect the electrocardiographic waveform, the feature amount of the electrocardiographic waveform can be accurately extracted, and the estimation accuracy of the biological state is also improved. In addition, even in a case where it is difficult to search the electrocardiographic waveform from only the electrocardiographic waveform, it is possible to accurately analyze the electrocardiographic waveform by searching the electrocardiographic waveform with reference to the second reference point based on the feature amount of the pulse waveform.

The biological state estimation device is only required to estimate the biological state on the basis of at least the feature amount of the electrocardiographic waveform among the feature amount of the electrocardiographic waveform and the feature amount of the pulse waveform. The biological state estimation device may estimate the biological state from only the feature amount of the electrocardiographic waveform, or may estimate the biological state on the basis of both the feature amount of the electrocardiographic waveform and the feature amount of the pulse waveform.

In the present invention,
the biological state estimation device may include a pulse waveform quality determination unit which determines whether or not the pulse waveform has a quality of being able to extract the feature amount of the pulse waveform, and
in a case where it is determined that the pulse waveform has the quality of being able to extract the feature amount of the pulse waveform, the pulse wave feature amount extraction unit may extract the feature amount of the pulse waveform.

As described above, in a case where it is determined whether or not the pulse waveform has the quality of being able to extract the feature amount of the pulse waveform, and the pulse waveform has such a quality, the second reference point is set on the basis of the extracted feature amount of the pulse waveform. Thus, even in a case where there is noise that is likely to affect the electrocardiographic waveform but is unlikely to affect the pulse waveform, the feature amount of the electrocardiographic waveform can be accurately extracted.

In the present invention,
the pulse wave feature amount extraction unit may extract a rise of the pulse waveform, and
the second search reference point setting unit may set a rising time of the pulse waveform as the second reference point.

As the feature amount of the pulse waveform, various feature amounts can be adopted, but in this way, the second reference point can be set by using the rise of the pulse waveform as the feature amount of the pulse waveform.

In the present invention,
the pulse wave feature amount extraction unit may extract a peak of the pulse waveform, and
the second search reference point setting unit may set a time of the peak of the pulse waveform as the second reference point.

In this way, the second reference point can be set by using the peak of the pulse waveform as the feature amount of the pulse waveform.

In the present invention,
the pulse wave feature amount extraction unit may extract a peak in a first differential waveform calculated from the pulse waveform, and
the second search reference point setting unit may set, as the second reference point, a time of the peak in the first differential waveform calculated from the pulse waveform.

In this way, the second reference point can be set by using, as the feature amount of the pulse waveform, the peak in the first differential waveform calculated from the pulse waveform.

In the present invention,
the pulse wave feature amount extraction unit may extract a peak in a second differential waveform calculated from the pulse waveform, and
the second search reference point setting unit may set, as the second reference point, a time of the peak in the second differential waveform calculated from the pulse waveform.

In this way, the second reference point can be set by using, as the feature amount of the pulse waveform, the peak in the second differential waveform calculated from the pulse waveform. As described above, as the pulse wave feature amount, the rise of the pulse waveform, the peak and the bottom of the pulse waveform, the peak of the first derivative or the second derivative of the pulse waveform, or the like can be adopted, but the pulse wave feature amount is not limited thereto.

In the present invention,
the electrocardiographic feature amount extraction unit may extract the feature amount of the electrocardiographic waveform from a synchronized summation average waveform obtained by synchronizing electrocardiographic waveforms for each beat specified on the basis of the second reference point set by the second search reference point setting unit and calculating a summation average.

When the feature amount of the electrocardiographic waveform is extracted, various methods can be adopted, and in a case such as noise of a myoelectric potential, the noise is canceled to obtain a clear synchronized summation waveform by synchronizing electrocardiographic waveforms for each beat and calculating a summation average. Therefore, the electrocardiographic feature amount can be accurately extracted by searching the synchronized summation average waveform.

In the present invention,
the biological state estimation device may include a display unit which displays information, and
the synchronized summation average waveform may be displayed on the display unit.

In this way, even in a case where noise such as a myoelectric potential is superimposed, a clear electrocardiographic waveform from which the noise is canceled can be displayed to the user.

In the present invention,
a blood pressure value may be estimated as the biological state.

In this way, it is possible to provide the biological state estimation device capable of accurately estimating the blood pressure value.

In the present invention,
a heart rate may be estimated as the biological state.

In this way, it is possible to provide the biological state estimation device capable of accurately estimating the heart rate.

### Advantageous Effects of Invention

According to the present invention, it is possible to provide a technique capable of searching an electrocardiographic waveform on the basis of a feature amount of a pulse waveform to accurately extract a feature amount of the electrocardiographic waveform even in a case where there is a noise that is likely to affect the electrocardiographic waveform.

### BRIEF DESCRIPTION OF DRAWINGS

FIGS. 1A and 1B are diagrams illustrating an external appearance of a biological state estimation device according to an embodiment.

FIG. 2 is a diagram for explaining a feature of the biological state estimation device according to the embodiment.

FIG. 3 is a diagram illustrating a hardware configuration of the biological state estimation device according to the embodiment.

FIG. 4 is a diagram illustrating a software configuration of the biological state estimation device according to the embodiment.

FIG. 5 is a diagram illustrating a search example of an electrocardiographic feature amount according to the embodiment.

FIG. 6 is a diagram illustrating another example of the search for the electrocardiographic feature amount according to the embodiment.

FIG. 7 is a diagram for explaining the search for the electrocardiographic feature amount according to the embodiment.

FIGS. 8A and 8B are diagrams for explaining the search for the electrocardiographic feature amount according to the embodiment.

FIGS. 9A and 9B are diagrams for explaining the search for the electrocardiographic feature amount according to the embodiment.

FIG. 10 is a flowchart for explaining a procedure of searching for an electrocardiogram according to the embodiment.

### DESCRIPTION OF EMBODIMENTS

Hereinafter, specific embodiments of the present invention will be described on the basis of the drawings.

### First Embodiment

Hereinafter, an embodiment of an embodiment of the present invention will be described. However, the dimensions, materials, shapes, relative arrangements, and the like of the components described in this embodiment are not intended to limit the scope of the present invention only to them unless otherwise specified.

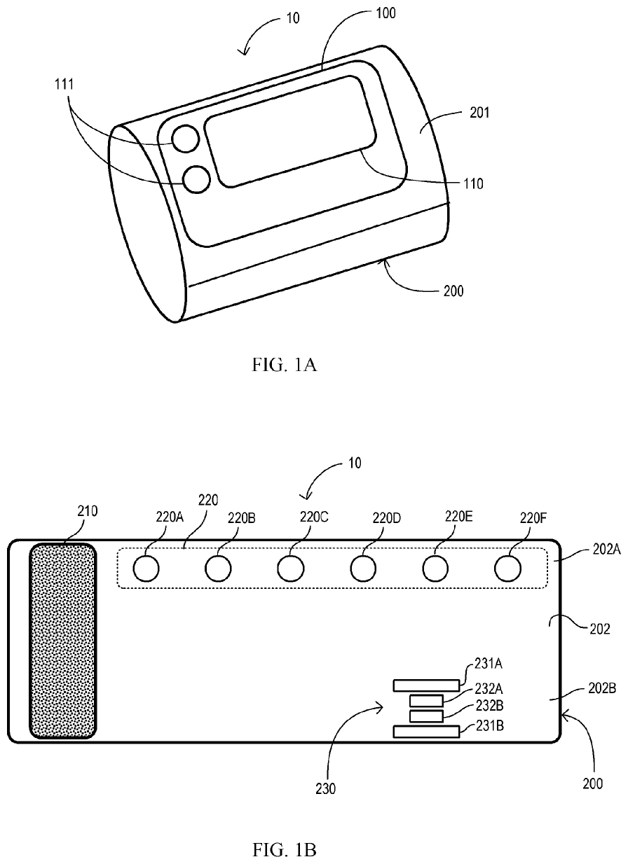

FIGS. 1A and 1B are external views of a biological state estimation device 10 according to the present embodiment. FIG. 1A illustrates an external appearance of a state where a belt 200 is wound and worn, and FIG. 1B is a diagram of the belt 200 in a developed state as viewed from an inner circumferential surface 202 side.

The biological state estimation device 10 mainly includes a main body 100 and the belt 200. The main body 100 is provided on an outer circumferential surface 201 of the belt 200, a control unit 101 and the like described later are accommodated, and a display unit 110 and an operation unit 111 are provided on the outer surface. On the inner circumferential surface 202 of the belt 200, one end in a longitudinal direction is provided with a hook-and-loop fastener 210. The hook-and-loop fastener on the other side to be engaged with the hook-and-loop fastener 210 at the time of wearing is provided on the outer circumferential surface 201 of the belt 200. Electrode groups 220A to 220F for electrocardiographic waveform measurement are arranged at equal intervals in the longitudinal direction at one edge portion 202A in a width direction of the inner circumferential surface 202 of the belt 200. An electrocardiographic sensor 220 including the electrode groups 220A to 220F is arranged at the edge portion 202A which is a shoulder side when worn on an upper arm. Electrodes 231A, 232A, 232B, and 231B constituting the sensor unit 230 of a pulse wave sensor are arranged at an end portion 202B, which on the other side in the width direction, of the inner circumferential surface 202 of the belt 200. The electrode 231A and the electrode 231B are electrodes for energizing the upper arm, and the electrode 232A and the electrode 232B are electrodes for detecting a voltage. The sensor unit 230 is provided at a position which is an elbow side when worn on the upper arm. When worn on the upper arm, the sensor unit 230 is arranged with the electrodes 231A, 232A, 232B, and 231B in order from the shoulder side to the elbow side along a brachial artery.

### (Features of Biological State Estimation Device)

The biological state estimation device 10 according to the present embodiment searches the electrocardiographic waveform (electrocardiogram) and the pulse waveform (pulse wave signal) acquired through the electrocardiographic sensor 220 and the sensor unit 230 of the pulse wave sensor described above, extracts the feature amount (electrocardiographic feature amount) of the electrocardiographic waveform and the feature amount (pulse wave feature amount) of the pulse waveform, and estimates a biological state such as a blood pressure value on the basis of at least the electrocardiographic feature amount among the electrocardiographic feature amount and the pulse wave feature amount. In a case where the electrocardiogram has a quality of being able to extract the electrocardiographic feature amount, and the pulse wave signal has a quality of being able to extract the pulse wave feature amount, it is possible to search each of the electrocardiogram and the pulse wave signal to extract the electrocardiographic feature amount and the pulse wave feature amount with high accuracy, and thus, it is possible to accurately estimate the biological state such as the blood pressure value based on these.

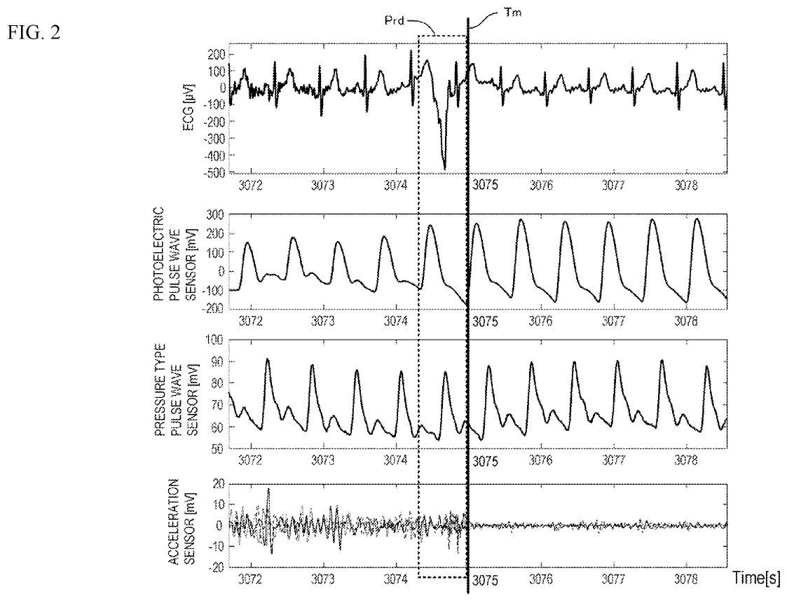

Noise may be superimposed on the electrocardiogram and the quality of the electrocardiogram may deteriorate depending on the wearing state of the biological state estimation device 10, the body motion, the posture, and the like of the user. It is difficult to accurately extract the electrocardiographic feature amount from only such an electrocardiogram having a low quality. At this time, depending on a cause of noise generated in the electrocardiogram, the electrocardiogram may be affected, but the pulse wave signal may not be affected or may be less affected. FIG. 2 is a graph illustrating a relationship between such an electrocardiogram and a pulse wave signal. Here, an uppermost part illustrates the electrocardiogram, a second part from the top illustrates the pulse wave signal detected by the photoelectric pulse wave sensor, a third part from the top illustrates the pulse wave signal detected by the pressure type pulse wave sensor, and a lowermost part illustrates the output signals (the output signals in three axial directions are indicated by a solid line, a broken line, and a dotted line) of the acceleration sensor. As also indicated in the output signals of the acceleration sensor, FIG. 2 illustrates waveforms acquired in a state where, with a time Tm indicated by a vertical solid line as a boundary, there is body motion of a user wearing the biological state estimation device 10 before the time Tm and there is no body motion after the time Tm. For this reason, the electrocardiogram of the uppermost part has a waveform in which a myoelectric potential is superimposed before the time Tm but has a waveform in which the myoelectric potential is not superimposed after the time Tm, and the waveform of the electrocardiogram significantly differs with the time Tm as a boundary. On the other hand, no significant change is observed in the pulse wave signal by any of the pulse wave sensors before and after the time Tm. In addition, since a large body motion has occurred in a period Prd indicated by a broken line, the waveform of the electrocardiogram is significantly disturbed, but no disturbance is observed in any of the pulse wave signals.

Due to such characteristics of the electrocardiogram and the pulse wave signal, even in a case where the electrocardiogram has a quality of being unable to extract the electrocardiographic feature amount from only the electrocardiogram, when the pulse wave signal has a quality of being able to extract the pulse wave feature amount, it is possible to set a search reference point for extracting the electrocardiographic feature amount by using the pulse wave feature amount as described later. Therefore, the electrocardiographic feature amount can be accurately extracted, and the biological state such as the blood pressure value can also be accurately estimated.

### (Hardware Configuration)

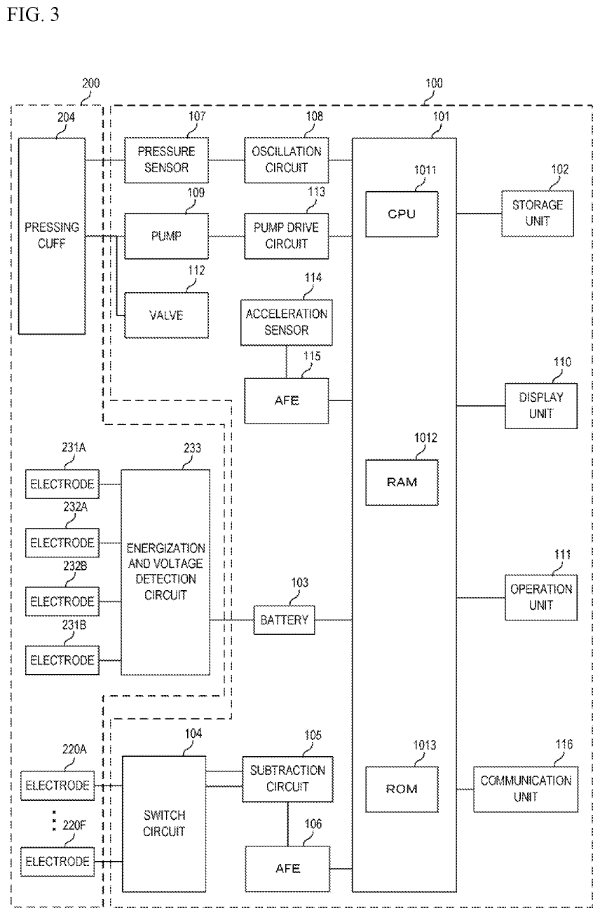

FIG. 3 illustrates a hardware configuration diagram of the biological state estimation device 10 according to the present embodiment. The biological state estimation device 10 mainly includes the main body 100 and the belt 200.

As described above, the electrode groups 220A to 220F for measuring the electrocardiogram are arranged on the inner circumferential surface of the belt 200.

The sensor unit 230 of the pulse wave sensor for measuring a pulse wave is arranged on the inner circumferential surface of the belt 200. The sensor unit 230 includes a pair of electrodes 231A and 231B for energizing the body of the user and a pair of electrodes 232A and 232B for detecting a voltage. In addition, the belt 200 is provided with an energization and voltage detection circuit 233 that energizes between the electrodes 231A and 231B of the sensor unit 230 and detects a voltage between the electrodes 232A and 232B. The energization and voltage detection circuit 233 may be provided in the main body 100.

In addition, the belt 200 includes a bag-shaped pressing cuff 204 that can store a fluid.

The main body 100 is provided with a control unit 101, a storage unit 102, a battery 103, a switch circuit 104, a subtraction circuit 105, an analog front end (AFE) 106, a pressure sensor 107, an oscillation circuit 108, a pump 109, a display unit 110, an operation unit 111, a valve 112, a pump drive circuit 113, an acceleration sensor 114, an AFE 115, and a communication unit 116.

The control unit 101 includes a central processing unit (CPU) 101I, a random access memory (RAM) 1012, a read only memory (ROM) 1013, and the like, and controls each component to realize various functions described later. The storage unit 102 is, for example, an auxiliary storage device such as a semiconductor memory or a hard disk drive (HDD), and stores a program executed by the control unit 101, setting data necessary for executing the program, a measurement result, and the like. A part or all of the program may be stored in the ROM 1013.

The battery 103 supplies power to the control unit 101 and the like. The battery 103 can be configured by, for example, a rechargeable battery.

Each of the electrodes 220A to 220F included in the electrocardiographic sensor 220 is connected to an input terminal of the switch circuit 104. Two output terminals of the switch circuit 104 are connected to two input terminals of the subtraction circuit 105, respectively. The switch circuit 104 receives a switch signal from the control unit 101, and connects two electrodes designated by the switch signal to the subtraction circuit 105. The subtraction circuit 105 subtracts, from the potential input from one input terminal, the potential input from the other input terminal. The subtraction circuit 105 outputs, to the AFE 106, a potential difference signal indicating a potential difference between the two connected electrodes. The subtraction circuit 105 is, for example, an instrumentation amplifier. The AFE 106 includes, for example, a low-pass filter (LPF), an amplifier, and an analog-to-digital (AD) converter. The potential difference signal is filtered by the LPF, amplified by the amplifier, and converted into a digital signal by the AD converter. The potential difference signal converted into the digital signal is output to the control unit 101. The control unit 101 acquires, as the electrocardiogram, potential difference signals acquired in time series from the AFE 106.

The energization and voltage detection circuit 233 causes a high-frequency constant current to flow between the electrodes 231A and 231B. For example, the frequency of the current is 50 kHz, and the current value is 1 mA. In a state where the electrodes 23A and 231B are energized, the energization and voltage detection circuit 233 detects a voltage between the electrodes 232A and 232B and generates a detection signal. The detection signal indicates a change in electrical impedance due to a pulse wave propagating through the portion of the artery where the electrodes 232A and 232B face each other. The energization and voltage detection circuit 233 performs signal processing including rectification, amplification, filtration, and AD conversion on the detection signal, and outputs the detection signal to the control unit 101. The control unit 101 acquires, as pulse wave signals, the detection signals output in time series from the energization and voltage detection circuit 233.

The acceleration sensor 114 detects accelerations of three axes in X, Y, and Z directions, and outputs a detection signal to the AFE 115. The AFE 115 includes, for example, an amplifier and an AD converter. The detection signal of the acceleration sensor 114 is amplified by the amplifier and converted into a digital signal by the AD converter. The detection signal converted into the digital signal is output to the control unit 101. The control unit 101 acquires, as triaxial acceleration signals, the detection signals acquired in time series from the AFE 115.

The pressure sensor 107 is connected to the pressing cuff 204 via a pipe, and the pump 109 and the valve 112 are connected to the pressing cuff 204 via a pipe. The pipe may be one common pipe. The pump 109 is, for example, a piezoelectric pump, and supplies air as a fluid to the pressing cuff 204 through a pipe in order to increase the pressure of the pressing cuff 204. The valve 112 is mounted on the pump 109, and the opening/closing thereof is controlled in accordance with the operation state of the pump 109. When the valve 112 is in an open state, the pressing cuff 204 communicates with the atmosphere, and the air in the pressing cuff 204 is discharged to the atmosphere. Note that the valve 112 has a function of a check valve, and does not allow backflow of air. The pump drive circuit 113 drives the pump 109 on the basis of the control signal received from the control unit 101.

The pressure sensor 107 detects a pressure (also referred to as a cuff pressure) in the pressing cuff 204 and generates an electric signal indicating the cuff pressure. The cuff pressure is, for example, a pressure based on the atmospheric pressure. The pressure sensor 107 is, for example, a piezoresistive pressure sensor. The oscillation circuit 108 oscillates on the basis of the electric signal from the pressure sensor 107, and outputs, to the control unit 101, a frequency signal having a frequency corresponding to the electric signal. Herein, the output of the pressure sensor 107 is used to control the pressure in the pressing cuff 204 and is also used to calculate the blood pressure value (including a systolic blood pressure (maximum blood pressure) and a diastolic blood pressure (minimum blood pressure)) by an oscillometric method.

Although an example in which the control unit 101 includes one CPU 101I has been described, the configuration of the control unit 101 is not limited thereto and may include a plurality of processors. In addition, the biological state estimation device 10 may include the communication unit 116 for communicating with an external device such as a mobile terminal (for example, a smartphone) of the user. As a communication method, an appropriate wired and/or wireless method can be adopted. Depending on the communication method, the communication unit 116 includes a wired communication module and/or a wireless communication module. As the wireless communication method, for example, Bluetooth (registered trademark), Bluetooth Low Energy (BLE), or the like can be adopted.

The operation unit 111 is an input device by which the user inputs an instruction to the biological state estimation device 10, and includes, for example, a button or the like.

The display unit 110 is a display device for displaying a measurement result or a message to the user, and can be configured by, for example, a liquid crystal display device (LCD) or an organic light emitting diode (OLED) display.

### (Software Configuration)

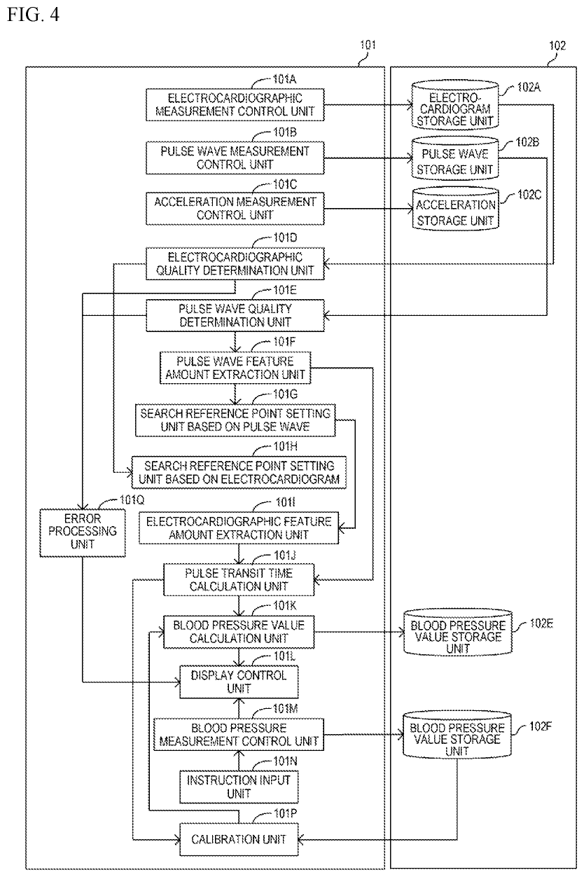

FIG. 4 illustrates a software configuration of the biological state estimation device 10 according to the present embodiment. An electrocardiographic measurement control unit 101A, a pulse wave measurement control unit 101B, an acceleration measurement control unit 101C, an electrocardiographic quality determination unit 101D, a pulse wave quality determination unit 101E, a pulse wave feature amount extraction unit 101F, a search reference point setting unit based on a pulse wave 101G, a search reference point setting unit based on an electrocardiogram 101H, an electrocardiographic feature amount extraction unit 101I, a pulse transit time calculation unit 101J, a blood pressure value calculation unit 101K, a display control unit 101L, a blood pressure measurement control unit 101M, an instruction input unit 101N, a calibration unit 101P, and an error processing unit 101Q illustrated in FIG. 4 are realized by the control unit 101 executing a program stored in a storage unit 192. An electrocardiogram storage unit 102A, the pulse wave storage unit 102B, the acceleration storage unit 102C, a blood pressure value storage unit 102E, and a blood pressure value storage unit 102F are realized by the storage unit 102.

The electrocardiographic measurement control unit 101A controls the switch circuit 104 to acquire an electrocardiogram. Specifically, the electrocardiographic measurement control unit 101A generates a switch signal for selecting two electrodes of six electrodes 220A to 220F, and outputs the switch signal to the switch circuit 104. The electrocardiographic measurement control unit 101A acquires potential difference signals obtained by using the selected two electrodes, and stores, as the electrocardiogram, time-series data of the acquired potential difference signals in the electrocardiogram storage unit 102A.

When the user wears the biological state estimation device 10 on the upper arm, the electrocardiographic measurement control unit 101A determines an electrode pair optimal for acquiring an electrocardiogram. For example, the electrocardiographic measurement control unit 101A acquires an electrocardiogram for each of all the electrode pairs, and determines an electrode pair, which provides an electrocardiogram having the largest amplitude of an R wave, as an optimal electrode pair. Thereafter, the electrocardiographic measurement control unit 101A measures the electrocardiogram by using the optimum electrode pair.

The pulse wave measurement control unit 101B controls the energization and voltage detection circuit 233 in order to acquire a pulse wave signal. Specifically, the pulse wave measurement control unit 101B instructs the energization and voltage detection circuit 233 to cause a current to flow between the electrodes 231A and 231B, and acquires a detection signal indicating a voltage between the electrodes 232A and 232B detected in a state where the current flows between the electrodes 231A and 231B. The pulse wave measurement control unit 101B causes the pulse wave storage unit 102B to store time-series data of the detection signal as a pulse wave signal.

The acceleration measurement control unit 101C acquires the accelerations of three axes output from the acceleration sensor 114 at a predetermined cycle, and causes the acceleration storage unit 102C to store the accelerations as an acceleration signal.

The electrocardiographic quality determination unit 101D determines whether or not the electrocardiogram stored in the electrocardiogram storage unit 102A has a quality of being able to extract the electrocardiographic feature amount from only the electrocardiogram. The following method can be used to determine the quality of the electrocardiogram. For example, the quality of the electrocardiogram can be determined by determining the quality of the contact state of the electrode according to a criterion such as whether the electrocardiographic waveform is constant. In addition, it is also possible to acquire the triaxial acceleration signal from the acceleration storage unit 102C and determine the quality of the electrocardiogram on the basis of whether or not the acceleration signal is equal to or less than a predetermined threshold value. In addition, the quality of the electrocardiogram can be determined by the presence or absence of mixing of the electromyogram. Since irregular and fine vibrations are observed when the electromyogram is mixed in the electrocardiogram, whether or not the electromyogram is mixed can be determined by detecting the presence or absence of such irregular and fine vibrations. For the determination of the quality of the electrocardiogram, any one of the above-described methods may be used, or any two or three or all of the above-described methods may be used. In addition, the determination of the quality of the electrocardiogram is not limited thereto, and other known methods can be adopted.

The pulse wave quality determination unit 101E determines whether or not the pulse wave signal stored in the pulse wave storage unit 102B has the quality of being able to extract the pulse wave feature amount. The following method can be used to determine the quality of the pulse wave signal. For example, the quality of the pulse wave signal can be determined by determining the quality of the contact state of the electrode according to a criterion such as whether the pulse wave signal is constant. In addition, it is also possible to acquire the triaxial acceleration signal from the acceleration storage unit 102C and determine the quality of the pulse wave signal on the basis of whether or not the acceleration signal is equal to or less than a predetermined threshold value. In addition, the quality of the pulse wave signal can be determined on the basis of whether or not the amplitude value of one beat of the pulse wave signal stored in the pulse wave storage unit 102B is equal to or less than a predetermined threshold value. For the determination of the quality of the pulse wave signal, any one of the above-described methods may be used, or any two or three or all of the above-described methods may be used. In addition, the determination of the quality of the pulse wave signal is not limited thereto, and other known methods can be adopted.

The search reference point setting unit based on the electrocardiogram 101H sets a reference point for searching the electrocardiogram when the electrocardiographic feature amount extraction unit 101I extracts the feature amount from the waveform of the electrocardiogram. For example, a peak point corresponding to the R wave can be used as the reference point. The reference point is not limited thereto, and a peak point corresponding to a Q wave, a peak point for an S wave, or the like may be used as the reference point according to the purpose.

As described above, the electrocardiographic feature amount extraction unit 101I searches the electrocardiogram for the reference point set by the search reference point setting unit based on the electrocardiogram 101H, and extracts the electrocardiographic feature amount. In addition to the R wave, various feature amounts such as a P wave, a QRS wave, a T wave, a PQRST wave, a J wave, a PQ interval, a QT interval, and a QRS time are targeted as the electrocardiographic feature amount.

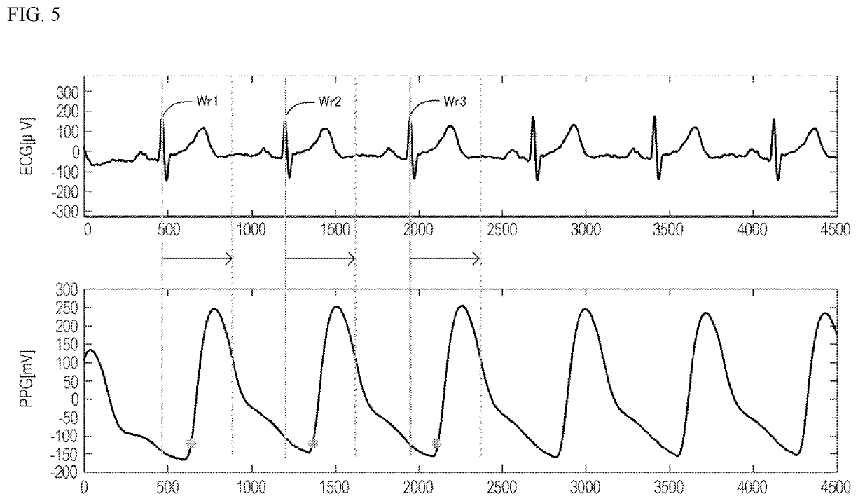

FIG. 5 illustrates an example of a case where the quality of the electrocardiogram is excellent and the electrocardiographic feature amount can be extracted from only the electrocardiogram. The upper part of FIG. 5 illustrates an example of the electrocardiogram read from the electrocardiogram storage unit 102A, and the lower part of FIG. 5 illustrates an example of the pulse wave read from the pulse wave storage unit 102B. When the quality of the electrocardiogram is excellent and the peak point corresponding to the R wave is set as the reference point, the electrocardiographic waveform is searched, peak points Wr1, Wr2, and Wr3 corresponding to the R wave are detected, and the time is specified. In this manner, a heartbeat time as the electrocardiographic feature amount is extracted as an interval between peaks corresponding to the R wave. A heart rate as the biological state can be estimated on the basis of the heartbeat time acquired in this manner. In addition, it is possible to search the electrocardiogram as indicated by an arrow with reference to the peak points Wr1, Wr2, and Wr3 corresponding to the R wave, and to extract various electrocardiographic feature amounts as described above.

In a section in which an excellent electrocardiogram can be acquired, the electrocardiographic feature amount can be extracted from only the electrocardiogram, but in some cases, the pulse wave feature amount (for example, a rise of a pulse wave) may be searched for with reference to the electrocardiographic feature amount (herein, the time of the peak point of the R wave of the electrocardiogram, which is the heartbeat time).

In a case where the electrocardiographic quality determination unit 101D determines that the electrocardiogram does not have the quality of being able to extract the electrocardiographic feature amount from only the electrocardiogram, the pulse wave feature amount extraction unit 101F extracts the pulse wave feature amount from the pulse wave stored in the pulse wave storage unit 102B, and subsequently the search reference point setting unit based on the pulse wave 101G sets the reference point for searching the electrocardiogram. As the feature amount related to the stroke of the pulse wave, a rise, a bottom, and a peak of the pulse wave, or a peak of a first derivative or a peak of a second derivative of the pulse wave can be set, but the feature amount related to the stroke of the pulse wave is not limited thereto.

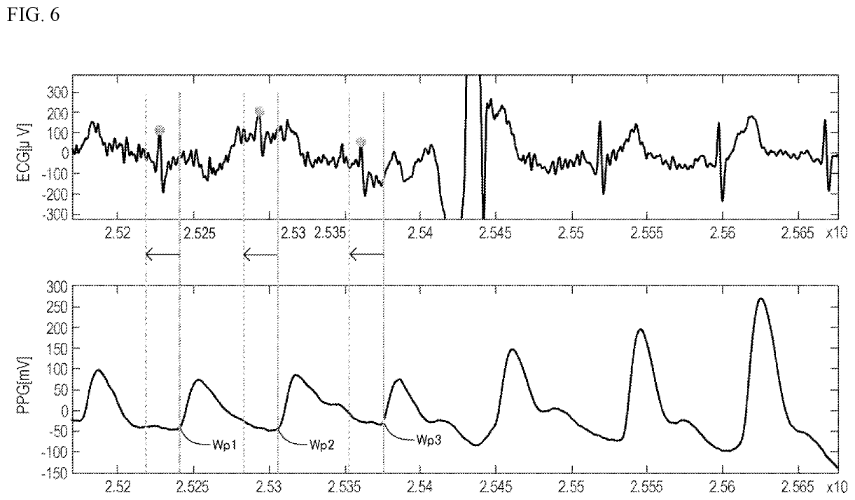

FIG. 6 illustrates an example of the electrocardiogram and the pulse wave signal in a case where it is determined that the quality of the electrocardiogram is not excellent and the electrocardiogram does not have the quality of being able to extract the electrocardiographic feature amount, but the pulse wave signal has the quality of being able to extract the pulse wave feature amount. The upper part of FIG. 6 illustrates an example of the electrocardiogram read from the electrocardiogram storage unit 102A, and the lower part of FIG. 6 illustrates an example of the pulse wave signal read from the pulse wave storage unit 102B. The pulse wave feature amount extraction unit 101F extracts rising points Wp1, Wp2, and Wp3, which are pulse wave feature amounts, from the pulse wave signal stored in the pulse wave storage unit 102B, and specifies the times thereof. The data of the pulse wave feature amount extracted in this manner is provided to the search reference point setting unit based on the pulse wave 101G.

In addition, the pulse wave feature amount extraction unit 101F extracts a pulse wave feature amount for estimating a blood pressure value to be described later.

The search reference point setting unit based on the pulse wave 101G sets a reference point for searching the electrocardiogram on the basis of the pulse wave feature amount extracted in this manner. When the rising point of the pulse wave signal is extracted, the search reference point setting unit based on the pulse wave 101G sets, as a reference point, the electrocardiogram time point corresponding to the time of the rising point of the pulse wave signal and gives the reference point to the electrocardiographic feature amount extraction unit 101I. The electrocardiographic feature amount extraction unit 101I searches for the feature amount of the electrocardiogram with reference to the search reference point set on the basis of the feature amount of the pulse wave received from the pulse wave feature amount extraction unit 101F. As described above, in a case where the rising points Wp1, Wp2, and Wp3 of the pulse wave signal and the times thereof are extracted as the pulse wave feature amounts, as indicated by arrows, the electrocardiographic feature amount extraction unit 101I searches the electrocardiogram within a predetermined time from the times of the rising points with reference to the rising points of the pulse wave signal, thereby detecting, for example, the peak points corresponding to the R wave and extracting as the electrocardiographic amount an interval between peaks corresponding to the R wave.

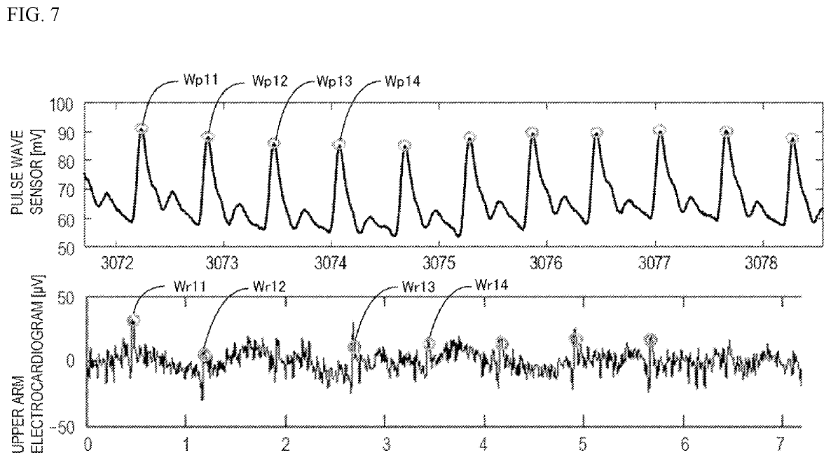

FIG. 7 illustrates another example of the electrocardiogram and the pulse wave signal in a case where it is determined that the electrocardiogram does not have the quality of being able to extract the electrocardiographic feature amount from only the electrocardiogram, but the pulse wave signal has the quality of being able to extract the pulse wave feature amount. Herein, in FIG. 7, the upper part is the pulse wave signal read from the pulse wave storage unit 102B, and the lower part is the electrocardiogram read from the electrocardiogram storage unit 102A. In this example, the electrocardiogram and the pulse wave were measured in a state where the user wearing the biological state estimation device 10 sat on a chair and raised his/her arm forward. When the electrocardiogram is measured for one arm, the electrocardiographic waveform itself detected by the electrocardiographic sensor 220 is small ( 1/10 or less of I induction), and the influence of the myoelectric potential is large.

In the example illustrated in FIG. 7, the pulse wave feature amount extraction unit 101F extracts peak points Wp11, Wp12, Wp13, and Wp14, or the like of the pulse wave.

On the basis of the data such as the peak point Wp11 of the pulse wave signal extracted in this manner, the search reference point setting unit based on the pulse wave 101G gives the electrocardiographic feature amount extraction unit 101I the times of the peak point Wp11 and the like of the pulse wave signal as a reference point for searching the electrocardiogram.

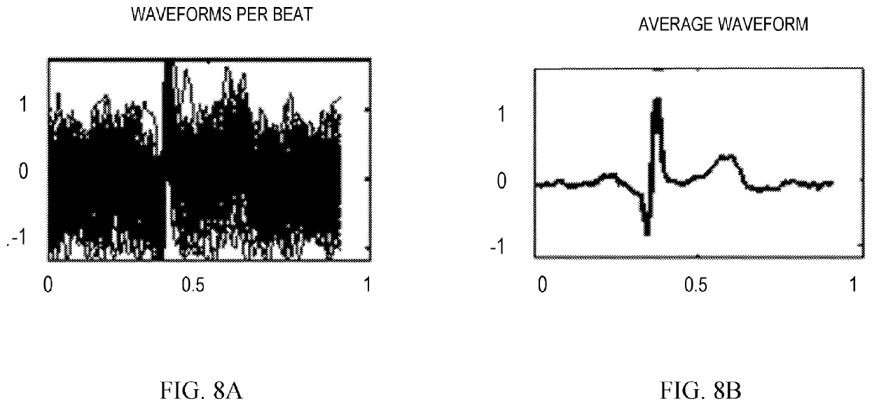

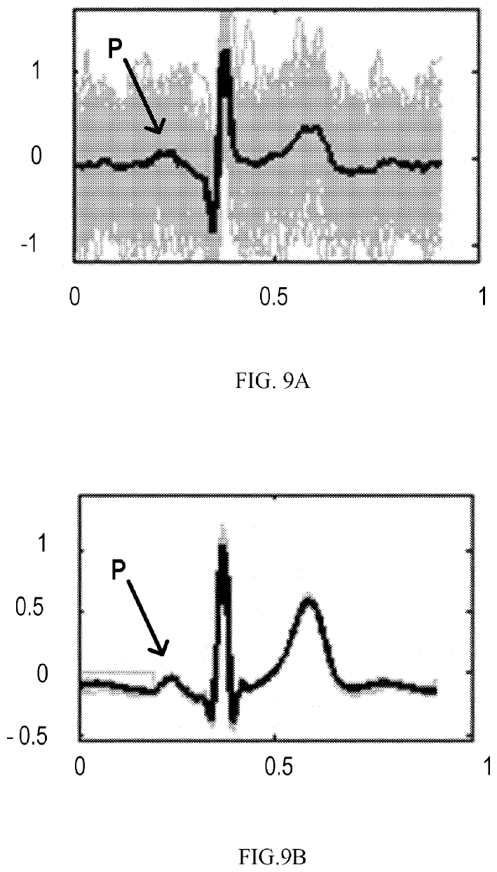

For example, the electrocardiographic feature amount extraction unit 101I searches the electrocardiogram with reference to the times of the peak point Wp11 and the like of the pulse wave signal, and extracts peak points Wr11, Wr12, Wr13, and Wr14, or the like corresponding to the R wave. As described above, even when the peak point corresponding to the R wave is extracted in the electrocardiogram, as illustrated in FIG. 7, what is obtained as the electrocardiogram is a noisy waveform on which the myoelectric potential is superimposed, and it is difficult to extract, from only the electrocardiogram, the electrocardiographic feature amount with reference to the peak point corresponding to the R wave. Therefore, when the synchronized summation average of the waveform for each beat as illustrated in FIG. 8A is taken with reference to the peak point corresponding to the R wave extracted as described above, a clear waveform (synchronized summation average waveform) illustrated in FIG. 8B is obtained. In FIG. 9A, the waveform for each beat is illustrated in gray, and the synchronized summation average waveform obtained by taking the synchronized summation average with reference to the peak point corresponding to the R wave as a reference is illustrated in black. In addition, FIG. 9B is a waveform of an electrocardiogram measured by I induction, which is illustrated for comparison. As the P wave can be clearly extracted in the electrocardiogram waveform illustrated in FIG. 9B, the P wave can be clearly extracted also in the electrocardiogram waveform based on the synchronized summation average illustrated in FIG. 9A, and thus other features of the electrocardiogram can be similarly extracted.

The pulse transit time calculation unit 101J calculates a pulse transit time on the basis of a time difference between the feature point of the electrocardiogram and the feature point of the pulse wave signal acquired from the electrocardiographic feature amount extraction unit 101I and the pulse wave feature amount extraction unit 101F. For example, the pulse transit time calculation unit 101J detects, from the electrocardiogram, the time of the peak point corresponding to the R wave, detects, from the pulse wave signal, the time of the rising point, and calculates, as the pulse transit time, a difference obtained by subtracting the time of the peak point from the time of the rising point.

The blood pressure value calculation unit 101K calculates a blood pressure value on the basis of the pulse transit time calculated by the pulse transit time calculation unit 101J and a calculation formula for calculating the blood pressure value from the pulse transit time. As the calculation formula for calculating the blood pressure value from the pulse transit time, a known calculation formula can be appropriately adopted, and thus the description thereof will be omitted. The blood pressure value calculated by the blood pressure value calculation unit 101K is stored in the blood pressure value storage unit 102E.

The instruction input unit 101N receives an instruction input from the user using the operation unit 111. For example, when the user performs an operation to instruct execution of blood pressure measurement, the instruction input unit 101N gives the blood pressure measurement control unit 101M an instruction to start blood pressure measurement.

The blood pressure measurement control unit 101M controls the pump drive circuit 113 to execute the blood pressure measurement. When receiving an instruction to start the blood pressure measurement from the instruction input unit 101N, the blood pressure measurement control unit 101M drives the pump 109 via the pump drive circuit 113. Accordingly, the supply of air to the pressing cuff 204 is started. The pressing cuff 204 is inflated, and the upper arm of the user is pressed. The blood pressure measurement control unit 101M monitors the cuff pressure by using the pressure sensor 107. In a pressurization process of supplying air to the pressing cuff 204, the blood pressure measurement control unit 101M calculates the blood pressure value by the oscillometric method on the basis of the pressure signal output from the pressure sensor 107. The blood pressure values include, but are not limited to, systolic blood pressure (SBP) and diastolic blood pressure (DBP). The blood pressure measurement control unit 101M stores the calculated blood pressure value in the blood pressure value storage unit 102F in association with the time information. The blood pressure measurement control unit 101M can calculate the heart rate simultaneously with the blood pressure value. When the calculation of the blood pressure value is completed, the blood pressure measurement control unit 101M causes the pump drive circuit 113 to stop the pump 109. Accordingly, air is discharged from the pressing cuff 204 through the valve 112. In a depressurization process of discharging the air from the pressing cuff 204 pressurized to a predetermined cuff pressure, the blood pressure measurement control unit 101M may calculate the blood pressure value by the oscillometric method.

The display control unit 101L controls the display unit 110 to display, on the display unit 110, a message for the user and a measurement result such as the blood pressure value and the heart rate.

The calibration unit 101P calibrates a blood pressure calculation formula on the basis of the pulse transit time obtained by the pulse transit time calculation unit 101J and the blood pressure value obtained by the blood pressure measurement control unit 101M. A correspondence relationship between the pulse transit time and the blood pressure value varies depending on an individual user and also varies depending on the wearing position of the biological state estimation device 10. Therefore, calibration is performed on the blood pressure calculation formula. As a method for calibrating the blood pressure calculation formula, a known method can be adopted, and thus the description thereof will be omitted.

In a case where the electrocardiographic quality determination unit 101D determines that the electrocardiogram does not have the quality of being able to extract the electrocardiographic feature amount from only the electrocardiogram, and the pulse wave quality determination unit 101E determines that the pulse wave signal does not have the quality of being able to extract the pulse wave feature amount, the error processing unit 101Q determines that there is an error and ends biological state estimation processing of the blood pressure value or the like. Then, the error processing unit 101Q causes the display control unit 101L to display, on the display unit 110, a message indicating that an error has occurred or a message.

In order to determine the qualities of the electrocardiogram and the pulse wave signal, not only the electrocardiogram and the pulse wave signal but also the acceleration detected by the acceleration sensor 114 can be used.

As described above, the biological state estimation device 10 according to the present embodiment sets the search reference point for searching the electrocardiogram for extracting the electrocardiographic feature amount on the basis of the electrocardiogram in a case where the electrocardiogram has the quality of being able to extract the electrocardiographic feature amount from only the electrocardiogram, but extracts the pulse wave feature amount detected by the sensor unit 230 of the pulse wave sensor to set the search reference point for searching the electrocardiogram in a case where the electrocardiogram has the quality of being unable to extract the electrocardiographic feature amount from only the electrocardiogram. As described above, since the electrocardiogram is searched on the basis of the pulse wave feature amount, even in a case where there is noise that is likely to affect the electrocardiogram but is unlikely to affect the pulse wave signal, it is possible to accurately extract a new feature amount, and further, to accurately estimate the biological state such as the blood pressure value.

Herein, the sensor unit 230 including the electrodes 231A, 231B, 232A, and 232B, the energization and voltage detection circuit 233, and the control unit 101 functioning as the pulse wave measurement control unit 101B correspond to the pulse waveform acquisition unit of the present invention. In addition, the electrode groups 230A to 230F, the switch circuit 104, the subtraction circuit 105, the AFE 106, and the control unit 101 functioning as the electrocardiographic measurement control unit 101A correspond to the electrocardiographic waveform acquisition unit of the present invention. The control unit 101 functioning as the electrocardiographic quality determination unit 101D corresponds to the electrocardiographic waveform quality determination unit of the present invention. The control unit 101 functioning as the pulse wave quality determination unit 101E corresponds to the pulse waveform quality determination unit of the present invention. The control unit 101 functioning as the search reference point setting unit based on the pulse wave 101G corresponds to the second search reference point setting unit of the present invention, and the reference point of the electrocardiogram search set by the search reference point setting unit based on the pulse wave 101G corresponds to the second reference point of the present invention. The control unit 101 functioning as the search reference point setting unit based on the electrocardiogram 101H corresponds to the first search reference point setting unit of the present invention, and the reference point of the electrocardiogram search set by the search reference point setting unit based on the electrocardiogram 101H corresponds to the first reference point of the present invention.

### (Electrocardiographic Feature Amount Extraction Method)

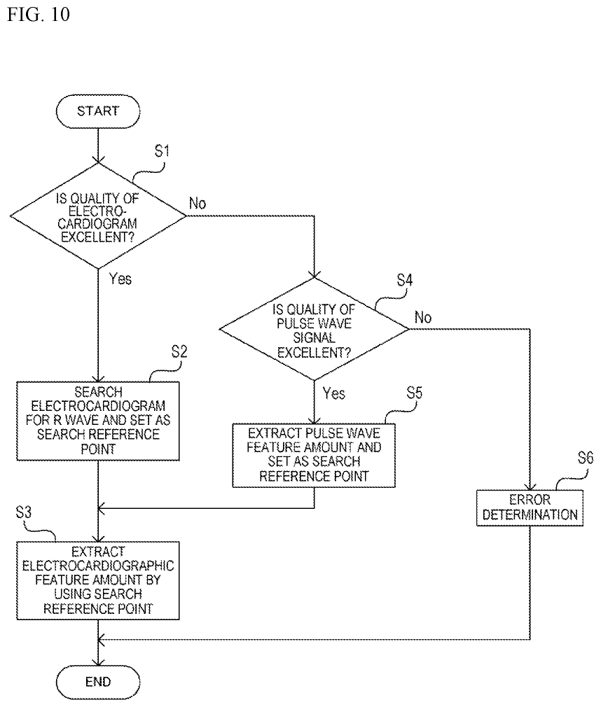

FIG. 10 is a flowchart illustrating an operation of extracting the electrocardiographic feature amount in the biological state estimation device 10 according to the present embodiment.

The following operation will be described assuming that the control unit 101 measures the electrocardiogram, the pulse waveform, and the acceleration and stores them in the storage unit 102.

First, in step S1, it is determined whether or not the quality of the electrocardiogram is excellent, that is, whether or not the electrocardiogram has the quality of being able to extract the electrocardiographic feature amount only on the basis of the electrocardiogram.

In a case where it is determined in step S1 that the quality of the electrocardiogram is excellent, the process proceeds to step S2.

In step S2, the peak point of the R wave in the electrocardiogram is searched for and set as the search reference point, and the process proceeds to step S3.

In a case where it is determined in step S1 that the quality of the electrocardiogram is not excellent, the process proceeds to step S4.

In step S4, it is determined whether or not the quality of the pulse wave signal is excellent, that is, whether or not the pulse wave signal has the quality of being able to extract the pulse wave feature amount on the basis of the pulse wave signal.

In a case where it is determined in step S4 that the quality of the pulse wave signal is excellent, the process proceeds to step S5.

In step S5, the feature amount of the pulse wave is extracted and set as the search reference point. For example, the rise of the pulse wave is extracted, the time of the rising point of the pulse wave is set as the search reference point, and the process proceeds to step S3.

Then, in step S3, the electrocardiographic feature amount is extracted by using the search reference point set in step S2 or step S5.

In a case where it is determined in step S4 that the quality of the pulse wave signal is not excellent, it is determined that there is an error, and the processing is ended. As error processing in a case where it is determined that there is an error, for example, information indicating that an error has occurred may be displayed on the display unit 110 to notify the user of the error. Depending on the cause of the error, a message such as “wear the belt 200 again” or “take a correct measurement posture” may be displayed on the display unit 110.

### REFERENCE SIGNS LIST

10 biological state estimation device
101 control unit
104 switch circuit
105 subtraction circuit
106 AFE
220 electrocardiographic sensor
230 sensor unit
233 energization and voltage detection circuit

1. A biological state estimation device which comprises:
an electrocardiographic waveform acquisition unit which acquires an electrocardiographic waveform;
an electrocardiographic feature amount extraction unit which searches the electrocardiographic waveform and extracts a feature amount of the electrocardiographic waveform;
a pulse waveform acquisition unit which acquires a pulse waveform; and
a pulse wave feature amount extraction unit which searches the pulse waveform and extracts a feature amount of the pulse waveform, and
which estimates a biological state on a basis of at least the feature amount of the electrocardiographic waveform among the feature amount of the electrocardiographic waveform and the feature amount of the pulse waveform,
the biological state estimation device further comprising:
an electrocardiographic waveform quality determination unit which determines whether or not the electrocardiographic waveform has a quality of being able to extract the feature amount of the electrocardiographic waveform from only the electrocardiographic waveform;
a first search reference point setting unit which sets a first reference point for searching the electrocardiographic waveform in a case where it is determined that the electrocardiographic waveform has the quality of being able to extract the feature amount of the electrocardiographic waveform; and
a second search reference point setting unit which sets a second reference point for searching the electrocardiographic waveform on a basis of the feature amount of the pulse waveform in a case where it is determined that the electrocardiographic waveform does not have the quality of being able to extract the feature amount of the electrocardiographic waveform,
wherein the electrocardiographic feature amount extraction unit searches the electrocardiographic waveform on a basis of the first reference point or the second reference point and extracts for each beat the feature amount of the electrocardiographic waveform.

2. The biological state estimation device according to claim 1, further comprising a pulse waveform quality determination unit which determines whether or not the pulse waveform has a quality of being able to extract the feature amount of the pulse waveform, wherein in a case where it is determined that the pulse waveform has the quality of being able to extract the feature amount of the pulse waveform, the pulse wave feature amount extraction unit extracts the feature amount of the pulse waveform.

3. The biological state estimation device according to claim 1, wherein
the pulse wave feature amount extraction unit extracts a rise of the pulse waveform, and
the second search reference point setting unit sets a rising time of the pulse waveform as the second reference point.

4. The biological state estimation device according to claim 1, wherein
the pulse wave feature amount extraction unit extracts a peak of the pulse waveform, and
the second search reference point setting unit sets a time of the peak of the pulse waveform as the second reference point.

5. The biological state estimation device according to claim 1, wherein
the pulse wave feature amount extraction unit extracts a peak in a first differential waveform calculated from the pulse waveform, and
the second search reference point setting unit sets, as the second reference point, a time of the peak in the first differential waveform calculated from the pulse waveform.

6. The biological state estimation device according to claim 1, wherein
the pulse wave feature amount extraction unit extracts a peak in a second differential waveform calculated from the pulse waveform, and
the second search reference point setting unit sets, as the second reference point, a time of the peak in the second differential waveform calculated from the pulse waveform.

7. The biological state estimation device according to claim 1, wherein a blood pressure value is estimated as the biological state.

8. The biological state estimation device according to claim 1, wherein a heart rate is estimated as the biological state.
